SEQUENCE LISTING (3 sequences)

SEQ ID NO: 1 - DNA, 1542 bp, Escherichia coli
aaattgaagagtttgatcatggctcagattgaacgctggcggcaggcctaacacatgcaagtcgaacggtaacaggaagcagcttgctgctttgctgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggagccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 2 - DNA, 19 bp, Artificial
  Synthetic eubac-F oligonucleotide
tcctacgggaggcagcagt

SEQ ID NO: 3 - DNA, 26 bp, Artificial
  Synthetic eubac-R oligonucleotide
ggactaccagggtatctaatcctgtt

(19) United States
(12) Patent Application Publication
(10) Pub. No.: US 2014/0088203 A1
(43) Pub. Date: Mar. 27, 2014

(54) METHOD FOR PREDICTING ABDOMINAL OBESITY

(71) Applicants: Jacques Amar, Toulouse (FR); Rémy Burcelin, Toulouse (FR)

(72) Inventors: Jacques Amar, Toulouse (FR); Rémy Burcelin, Toulouse (FR)

(73) Assignees: CHU de Toulouse, Toulouse Cedex 9 (FR); INSTITUT NATIONAL DE LA SANTE ET DE LA RECHERCHE MEDICALE (INSERM), paris (FR)

(21) Appl. No.: 14/008,661

(22) PCT Filed: Apr. 2, 2012
(86) PCT No.: PCT/EP12/55982

§ 371 Date: Dec. 6, 2013

(30) Foreign Application Priority Data
Mar. 31, 2011 (EP) 11305369.8

(51) Int. Cl. C12Q 1/68

(52) U.S. Cl. CPC C12Q 1/6883
USPC 514/789; 435/6.12

(57) ABSTRACT

The present invention relates to a method, in particular an in vitro method, for predicting a risk of onset of abdominal obesity in a subject, which method comprises the steps of: a1) measuring the concentration of bacterial 16S rDNA in a biological sample of said subject; and b) based on the result of the measurement in step a1), determining a risk of onset of abdominal obesity in the subject.

The present invention concerns a method for predicting abdominal obesity.

Obesity is reaching epidemic proportions in Western countries. In 2004 in the United States, 65% of adults were overweight or obese, 30% were obese, and 5% were morbidly obese. Obesity is associated with numerous cardiovascular diseases such as coronary heart disease, hypertension and type 2 diabetes. However, these complications are not observed in all obese patients. They are more particularly associated with abdominal obesity.

Abdominal obesity, or central obesity, is the accumulation of abdominal fat resulting in an increase in waist size. More than 60% of adult females in the United States have abdominal obesity and recent data suggest that the prevalence of abdominal obesity continues to increase. Current clinical guidelines recommend initiating weight loss treatment in women whose waist circumference is >88 cm (or body mass index of 25 to 29.9 kg/m²) and who suffer from two or more diseases among type 2 diabetes, cardiovascular disease, hypertension and dyslipidemia. Nevertheless, although abdominal fat decreases with weight loss, interventions to sustain long-term weight loss have not been identified.

There is thus a need for methods for specifically predicting abdominal obesity, in particular in order to determine an appropriate prevention treatment before abdominal fat reaches an abnormal level.

The causal role of the intestinal microbiota on weight gain has been demonstrated in experiments in which germ-free mice colonized with intestinal microbiota from genetically obese ob/ob mice gained more weight than their counterparts colonized with microbiota from lean animals (Turnbaugh et al. (2006) Nature 444:1027-1031). In humans, it has been shown that obesity was associated with phylum-level changes in the gut microbiota and reduced bacterial diversity (Turnbaugh et al. (2009) Nature 457:480-484). Furthermore, it has been demonstrated that gut microbiota affected energy balance by influencing the efficiency of calorie harvest from the diet and the way this harvested energy was used and stored. The role of bacterial components within blood in relation to weight has also been demonstrated: mice fed normal chow and chronically infused with a low dose of lipopolysaccharides (LPS) developed obesity, whereas mice carrying a deletion in the gene for CD14, a component from the principal receptor for bacterial LPS, did not (Cani et al. (2007) Diabetes 56:1761-1772). Nevertheless, these studies did not enable identifying early markers of abdominal obesity.

The present invention first arises from the unexpected finding by the inventors that the plasma concentration of bacterial 16S rDNA predicted the onset of abdominal obesity, but not of general obesity, in a large cohort of apparently healthy subjects.

The present invention thus relates to a method, in particular an in vitro method, for predicting a risk of onset of abdominal obesity in a subject, which method comprises the steps of:
a1) measuring the concentration of bacterial 16S rDNA in a biological sample of said subject; and
b) based on the result of the measurement in step a1), determining a risk of onset of abdominal obesity in the subject.

### DETAILED DESCRIPTION OF THE INVENTION

### Obesity

As used herein, the term “obesity”, “general obesity” or “overall obesity” refers to a medical condition in which excess body fat has accumulated to the extent that it may have an adverse effect on health, leading to reduced life expectancy and/or increased health problems. General obesity is typically determined by assessing the body mass index (BMI), a measurement which associates weight and height. In particular, people are defined as overweight if their BMI is between 25 kg/m² and 30 kg/m², and obese when it is greater than 30 kg/m².

In the context of the invention, the term “abdominal obesity”, “central obesity” or “belly fat” refers to obesity wherein there is a specific accumulation of abdominal fat resulting in an increase in waist size. Typically, in abdominal obesity, visceral fat, also known as organ fat or intra-abdominal fat, is located inside the peritoneal cavity, packed in between internal organs and torso, whereas, in general obesity, subcutaneous fat is found underneath the skin, and intramuscular fat is found interspersed in skeletal muscle.

Abdominal obesity is typically determined just by looking at the naked body, or more specifically by taking waist and hip measurements. The absolute waist circumference (>102 centimetres (40 inches) in men and >88 centimetres (35 inches) in women) and the waist-hip ratio (>0.9 for men and >0.85 for women) are both used as measures of abdominal obesity. Preferably, the expression “abdominal adiposity” according to the invention refers to a waist circumference of more than 102 cm in men or of more than 88 cm in women.

### Subject

In the context of the present invention, a “subject” denotes a human or non-human mammal, such as a rodent (rat, mouse, rabbit), a primate (chimpanzee), a feline (cat), or a canine (dog). Preferably, the subject is human. The subject according to the invention may be in particular a male or a female. In a particular embodiment of the invention, the subject according to the invention is a male subject.

Preferably, the subject according to the invention is 30-65 years old.

In a particular embodiment, the subject according to the invention does not suffer from abdominal obesity at the time of sampling.

In another particular embodiment, the subject according to the invention does not suffer from general obesity at the time of sampling.

In still another particular embodiment, the subject according to the invention is free of known obesity risk factors and/or known abdominal obesity risk factors.

As used herein, the expression “obesity and/or abdominal obesity risk factor” refers to a biological marker which is associated with the onset of general and/or abdominal obesity. Some general and/or abdominal obesity risk factors are well-known from the skilled person and include for example age, short sleep duration, early puberty, age at menarche, low activity level, sedentarity, smoking, alcohol intake, hypertension, hypertriglyceridemia, hyperglycemia, genetic and epigenetic factors, environmental factors, familial history of obesity, poor quality of life and poor dietary quality.

As used herein, the expression “short sleep duration” refers to sleep duration inferior to 6-7 hours.

As used herein, the expression “early puberty” refers to an onset of signs of puberty before age 7 or 8 in girls and age 9 for boys.

As used herein, the expression “low activity level” refers to the fact of exercising less than 3 times a week.

As used herein, the expression “sedentarity” or “sedentary life style” denotes a type of lifestyle with no or irregular physical activity.

As used herein, the expression “hypertension” also referred to as “high blood pressure”, “HTN” or “HPN”, denotes a medical condition in which the blood pressure is chronically elevated. In the context of the invention, hypertension is preferably defined by systolic/diastolic blood pressure of at least 140/90 mmHg or being on antihypertensive medication.

As used herein, the expression “hypertriglyceridemia” or “high blood levels of triglycerides” refers to a blood level of triglycerides superior to 250 mg/dl.

As used herein, the expression “hyperglycemia” or “high fasting glycemia” denotes a syndrome of disordered metabolism, resulting in a glycemia, in particular a fasting glycemia, of more than 6.1 mmol/l.

As used herein, the expression “quality of life” refers to the general well-being of individuals and societies. Typically, indicators of the quality of life include wealth, employment, built environment, physical and mental health, education, recreation and leisure time, and social belonging. The quality of life is preferably assessed using the Human Development Index (HDI), which combines measures of life expectancy, education, and standard of living.

As used herein, the expression “poor quality dietary” refers to a dietary with a low obesity-specific nutritional risk score (ONRS), as described for example in Wolongevicz et al. (2010) J. Obesity 2010:1-9. Such an ONRS includes typically the following components: total energy (kJ), energy density (kJ/g), carbohydrate (% energy), protein (% energy), total, monounsaturated, polyunsaturated and saturated fats (% energy), fiber (g/4184 kJ), calcium (mg/4184 kJ) and alcohol (% energy).

In a particular embodiment, the subject according to the invention is free of high fasting glycemia or of the metabolic syndrome.

As used herein, the expression “metabolic syndrome” refers to a multiplex risk factor for cardiovascular disease comprising the 6 following components: abdominal obesity, atherogenic dyslipidemia, raised blood pressure, insulin resistance with or without glucose intolerance, proinflammatory state and prothrombotic state. The metabolic syndrome is more specifically defined in Grundy et al. (2004) Circulation 109:433-438.

In another particular embodiment, the subject according to the invention does not have any infection. Accordingly, the subject according to the invention preferably displays a plasma baseline C reactive protein concentration lower than 10 mg/l and/or does not present an abundant leukocyturia and/or does not take antiviral therapy.

As used herein, the term “C reactive protein” or “CRP” refers to a protein which is a member of the class of acute-phase reactants, as its levels rise dramatically during inflammatory processes occurring in the body. As known from the skilled person, CRP is a 224-residue protein with a monomer molar mass of 25106 Da, encoded by the CRP gene.

As used herein, the term “leukocyturia” refers to the presence of leukocytes in the urine of the subject. In particular, an abundant leukocyturia corresponds to the presence of more than 10 leukocytes/mm³ in the urine.

### Bacterial 16S rDNA

In the context of the invention, the expressions “16S rDNA” and “16S ribosomal DNA” are used indifferently and refer to the gene encoding the 16S ribosomal RNA constituted of about 1500 nucleotides, which is the main component of the small prokaryotic ribosomal subunit (30S). 16S rDNA is highly conserved among bacteria. The reference Escherichia coli 16S rDNA gene sequence corresponds to SEQ ID NO: 1 (called rrsA). In the context of the invention, 16S rDNA refers to any sequence corresponding to SEQ ID NO: 1 in other bacterial strains.

### In Vitro Method for Predicting a Risk of Onset

The present invention concerns an in vitro method for predicting a risk of onset of abdominal obesity in a subject, which method comprises the steps of:

a1) measuring the concentration of bacterial 16S rDNA in a biological sample of said subject; and

b) based on the result of the measurement in step a1), determining a risk of onset of abdominal obesity in the subject.

As used herein, a “predicting method” or “method for predicting” refers to a method for determining whether an individual is likely to develop a disease.

As used herein, the expression “risk of onset” of a disease refers to the probability that a disease will appear in a studied subject, in particular within a given period of time.

Preferably, the concentration of bacterial 16S rDNA is measured by polymerase chain reaction (PCR), more preferably by quantitative PCR (qPCR), most preferably by real-time or real-time quantitative PCR(RT-PCR or RT-qPCR).

As used herein, “real-time PCR”, “real-time quantitative PCR”, “real-time polymerase chain reaction” or “kinetic polymerase chain reaction” refers to a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a sample. Two common methods of quantification are the use of fluorescent dyes that intercalate with double-stranded DNA, and modified DNA oligonucleotide probes that fluoresce when hybridized with a complementary DNA.

As used herein, the term “biological sample” means a substance of biological origin. Examples of biological samples include, but are not limited to, blood and components thereof such as serum, plasma, platelets, subpopulations of blood cells such as leucocytes, urine, saliva, fecal water and tissues such as adipose tissues, hepatic tissues, pancreatic tissues and the like. Preferably, a biological sample according to the present invention is a blood, serum, plasma, leucocytes, urine, adipose tissue or hepatic tissue sample. More preferably, the biological sample is selected from the group consisting of blood, serum and plasma sample. The biological sample according to the invention may be obtained from the subject by any appropriate means of sampling known from the skilled person.

Specifically, the present inventors demonstrated that the risk of onset of abdominal obesity in a subject was linearly associated with the bacterial 16S rDNA concentration in said subject. Accordingly, the higher the bacterial 16S rDNA concentration, the higher the risk of onset of abdominal obesity.

More particularly, the inventors determined that the adjusted odds ratio (adjusted on sex, baseline age, family history of diabetes, smoking status, hypertension, waist circumference, body mass index and fasting plasma glucose) for an increase of the logarithm of the standard deviation of 16S rDNA mean concentration (log(0.27)), was of 1.18 (with a 95% confidence interval of 1.03-1.34). Accordingly, typically, in the methods according to the invention, a subject, displaying an increase of log(0.27) of the 16S rDNA concentration, has 1.18 more risk of having abdominal obesity, the 16S rDNA concentration being preferably measured by real-time PCR, preferably using the universal forward and reverse primers eubac-F (5′-TCCTACGGGAGGCAGCAGT-3′ SEQ ID NO: 2) and eubac-R (5′-GGACTACCAGGGTATCTAATCCTGTT-3′ SEQ ID NO: 3), typically using the following reaction conditions for amplification of DNA: 95° C. for 10 min and 35 cycles of 95° C. for 15 s and 60° C. for 1 min.

In a particular embodiment, the method of predicting a risk of onset of abdominal obesity as defined above further comprises a step a2) of comparing the measured bacterial 16S rDNA concentration with a predetermined value.

Preferably, the predetermined value corresponds to the normal concentration of bacterial 16S rDNA.

As intended herein a “normal concentration” of bacterial 16S rDNA means that the concentration of 16S rDNA in the biological sample is within the norm cut-off values for that gene. The norm is dependant on the biological sample type and on the method used for measuring the concentration of 16S rDNA in the biological sample. In particular, the predetermined value is the mean concentration of bacterial 16S rDNA in a healthy population.

As used herein, a “healthy population” means a population constituted of subjects who have not previously been diagnosed with obesity or abdominal obesity or who do not display obesity risk factors or abdominal obesity risk factors as defined above. Subjects of a healthy population also do not otherwise exhibit symptoms of disease. In other words, such subjects, if examined by a medical professional, would be characterized as healthy and free of symptoms of disease.

Preferably, in the methods of the invention, it is further determined whether the measured concentration of bacterial 16S rDNA is increased compared to the predetermined value according to the invention. Still preferably, in the methods of the invention, it is further determined the level of increase of the measured concentration of bacterial 16S rDNA compared to the predetermined value according to the invention.

As used herein, the expression “level of increase” means the percentage of increase of the measured concentration of bacterial 16S rDNA compared to the predetermined value according to the invention or the number of fold of increase of the measured concentration of bacterial 16S rDNA compared to the predetermined value according to the invention.

The inventors specifically demonstrated that the increase of concentration of bacterial 16S rDNA in the biological sample of a subject compared to the predetermined value enabled predicting with a very high significance an increase of the risk of onset of abdominal obesity, but not of general obesity.

Moreover, the inventors demonstrated that the concentration of bacterial 16S rDNA enabled predicting the onset of abdominal obesity as soon as 9 years before the onset of the disease.

Accordingly, in preferred methods according to the invention, a measured concentration of bacterial 16S rDNA in the biological sample of the subject which is higher than the predetermined value is indicative of an increased risk of onset of abdominal obesity within 9 years from the sampling.

The invention will be further illustrated by the following example and figures.

### DESCRIPTION OF THE FIGURES

FIG. 1 shows a graphical representation of the relations between quartiles of 16S rDNA gene concentrations and obesity after nine years follow-up in the overall population, showing the percentage of cases of obesity according to the quartiles of 16S rDNA concentration (ng/μl), a case of obesity corresponding to BMI≧30 kg/m².

FIG. 2 shows a graphical representation of the relations between quartiles of 16S rDNA gene concentrations and abdominal obesity after nine years follow-up in the overall population, showing the percentage of cases of abdominal obesity according to the quartiles of 16S rDNA concentration (ng/μl), a case of abdominal obesity corresponding to BMI≧30 kg/m² and a waist circumference>102 cm for men, >88 cm for women.

### EXAMPLE

The following example demonstrates the predictive value of blood bacterial 16S rDNA on the onset of abdominal obesity, but not of general obesity.

### Methods

### Population

D.E.S.I.R. is a longitudinal cohort study of 5,212 adults aged 30-65 years at baseline. Participants were recruited in 1994-1996 from ten Social Security Health Examination centers in central-western France, from volunteers insured by the French national social security system (80% of the French population—any employed or retired person and their dependents are offered free periodic health examinations). Equal numbers of men and women were recruited in five-year age groups. All participants gave written informed consent, and the study protocol was approved by the CCPPRB (ComitéConsultatif de Protection des Personnes pour la Recherche Biomédicale) of the Hôpital Bicêtre (Paris, France). Participants were clinically and biologically evaluated at inclusion and at 3-, 6-, and 9-yearly follow-up visits. The inventors studied individuals without diabetes at baseline (defined by treatment for diabetes or fasting plasma glucose≧7.0 mmol/l) and obesity (body mass index≧30 kg/m²) and who had a known diabetes status at the year-9 examination, with measures of baseline 16S rDNA gene concentrations. They excluded those who were likely to have infection defined: C-reactive protein (CRP)>10 mg/l, abundant leucocyturia, taking antiviral therapy.

### Parameters Studied

Weight and height were measured in lightly clad participants and body mass index (BMI) was calculated. Waist circumference, the smallest circumference between the lower ribs and the iliac crests, was also measured. The examining physician noted the family history of diabetes and treatment for diabetes and hypertension were recorded. Hypertension was defined by systolic/diastolic blood pressure of at least 140/90 mmHg or being on antihypertensive medication. Smoking habits were documented in a self-administered questionnaire.

Presence of the metabolic syndrome according to the NCEP criteria (Expert Panel on Detection, Evaluation, and Treatment of High Blood Cholesterol in Adults (2001) JAMA 285:2486-2497) was recorded. Central adiposity was defined by a waist circumference>102 cm in men and >88 cm in women, high fasting glucose by ≧6.1 mmol/l.

### Biological Analyses

Blood was drawn after a 12-h fast. All biochemical measurements except insulin and bacterial DNA analysis were from one of four health center laboratories located in France at Blois, Chartres, La Riche, or Orléans. Fasting plasma glucose, measured by the glucose oxidase method, was applied to fluoro-oxalated plasma using a Technicon RA100 (Bayer Diagnostics, Puteaux, France) or a Specific or a Delta device (Konelab, Evry, France). HbA1c was measured by high-performance liquid chromatography, using a L9100 automated ion-exchange analyzer (Hitachi/Merck-VWR, Fontenay-sous-Bois, France) or by DCA 2000 automated immunoassay system (Bayer Diagnostics, Puteaux, France). Both glucose and HbA1c were standardized across laboratories. Insulin was measured centrally by a Micro particle Enzyme Immunoassay with the IMX automated analyser from Abbott. CRP was assayed by BNII nephelometer (Behring, Rueil Malmaison, France). Total cholesterol and triglycerides were measured by enzymatic methods.

16S rDNA Gene Quantification

Total DNA concentration was determined using the Quant-iT™ dsDNA Broad-Range Assay Kit (Invitrogen) and a procedure adapted by the genomic platform of the Genopole Toulouse Midi Pyrénées (http://genomique.genotoul.fr). The mean concentration was 121.1±208.3 ng/μl. Each sample was diluted ten-fold in Tris buffer EDTA. The DNA was amplified by realtime PCR (Stepone+; Applied Biosystems) in optical grade 96-well plates. The PCR reaction was performed in a total volume of 25 μL using the Power SYBR® Green PCR master mix (Applied Biosystems), containing 300 nM of each of the universal forward and reverse primers eubac-F (5′-TCCTACGGGAGGCAGCAGT-3′) (SEQ ID NO: 2) and eubac-R (5′-GGACTACCAGGGTATCTAATCCTGTT-3′) (SEQ ID NO: 3). The reaction conditions for amplification of DNA were 95° C. for 10 min and 35 cycles of 95° C. for 15 s and 60° C. for 1 min. The amplification step was followed by a melting curve step according to the manufacturer's instructions (from 60° C. to 90° C.) to determine the specificity of the amplification product obtained. The amount of DNA amplified was compared with a purified 16S rDNA from E. coli BL21 standard curve, obtained by real time PCR from DNA dilutions ranging from 0.001 to 10 ng/μL.

### Statistical Analyses

For statistical analysis, the 16S rDNA gene concentrations were log transformed, as the distribution was skewed, as were the levels of triglycerides, insulin and CRP.

Characteristics of subjects are shown as means, the standard deviation (SD) being indicated into brackets, or as n, the corresponding percentage in the study population being indicated into brackets. The t test was used to compare blood bacterial gene concentration as a continuous variable (logarithm) between subjects destined to become obese and those who did not, and between those who were and were not abdominally obese after 9 years of follow-up. Logistic regression was used to calculate the standardized odds ratios and the 95 percent confidence intervals for incident abdominal obesity, according to one SD of baseline 16S rDNA gene concentrations, as a continuous variable (logarithm). Adjustments were made for sex, baseline age, family history of diabetes, hypertension, waist circumference, BMI, smoking status, fasting plasma glucose. The relation with 16S rDNA gene concentrations (logarithm) was linear, as an additional squared term was not significant. Odds ratios were also calculated over risk-factor strata. The relation between quartiles of 16S rDNA gene concentrations and incident obesity and abdominal obesity study are shown.

SAS versions 9.1 and 9.2 were used for statistical analysis.

### Results

### Characteristics of the Studied Population

At baseline, among the 5212 participants in the D.E.S.I.R. study, 126 participants had diabetes, 474 were obese, 65 presented biological signs of infection or received antiviral therapy, 333 did not undergo 16S rDNA gene concentration determination and for 1146, diabetes status was not known at the end of the nine years, as they did not attend the year-9 examination. These volunteers were excluded from the analysis. The characteristics of study population are shown in Table 1.

TABLE 1
Baseline characteristics (mean (SD) and n (%)) of study population

| | N = 3280 | |
|---|---|---|
| Age | 47 | (10) |
| Women (%) | 1666 | (51) |
| Diabetes in family | 604 | (19) |
| Current smoker | 622 | (19) |
| BMI (kg/m²) | 23.9 | (2.8) |
| Waist (cm) | | |
| men | 88 | (8) |
| women | 75 | (8) |
| Hypertension (%)^{†} | 1055 | (32) |
| Glucose (mmol/l) | 5.26 | (0.52) |
| HBA1c (%) | 5.41 | (0.39) |
| Insulin (pmol/l)* | 42.58 | (22.46) |
| CRP (mg/l) | 1.64 | (1.48) |
| Total cholesterol (mmol/l) | 5.70 | (0.98) |
| Triglycerides (mmol/) | 1.09 | (0.71) |
| 16S rDNA gene (ng/μl) | 0.13 | (0.27) |

^{†}SBP ≧ 140 mmHg and/or DBP ≧ 90 mmHg and/or an antihypertensive treatment.

### Prediction of Abdominal Adiposity

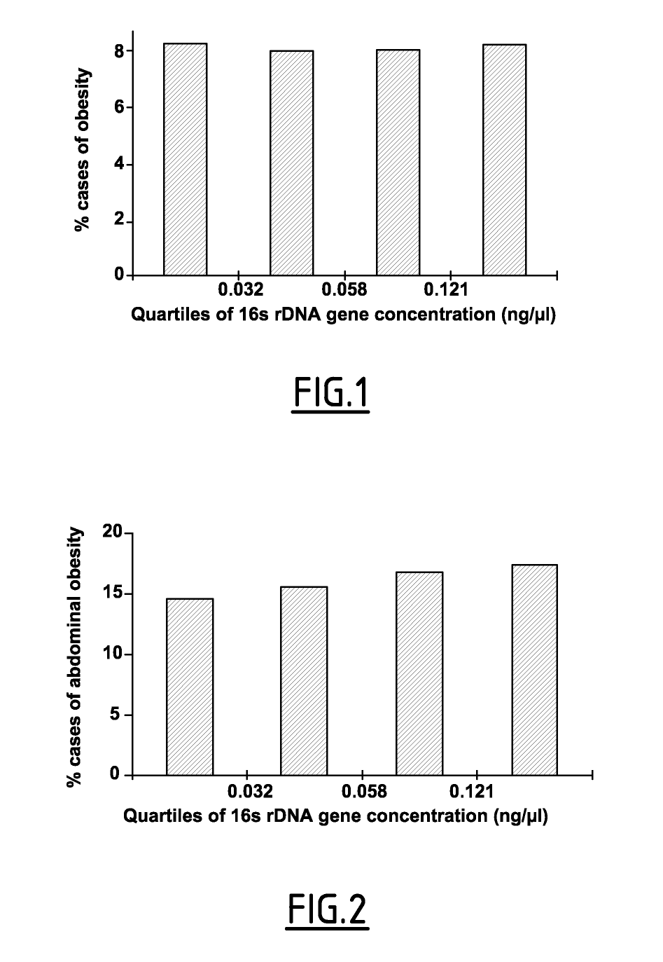

Incident cases of obesity were recorded and presented in percentage of incidence. No difference in blood bacterial gene concentration was observed according those with incident obesity. In contrast, mean 16S rDNA gene concentrations tended to be higher (n=485: 0.14±0.30 vs n=2795: 0.13±0.26, p=0.05) in those with abdominal adiposity at the end of nine years of follow-up. A graphical representation of these findings is shown in FIGS. 1 and 2. The same trend was seen for the incidence of abdominal adiposity in the 3088 individuals without abdominal adiposity at baseline.

The 16S rDNA gene concentration predicted the presence of abdominal adiposity at the end of nine years, after adjustment for confounders, with a standardized odds ratio of 1.18 (1.03 to 1.34), p=0.01.

In the subgroup of individuals free of abdominal adiposity at inclusion, the standardized odds ratio was 1.12 (0.98 to 1.27) p=0.09.

### CONCLUSION

The inventors showed, for the first time, that the concentration of a blood bacterial component, the 16S rDNA gene, predicts the presence of abdominal adiposity in a large sample of non obese subjects from a general population, after adjusting for traditional metabolic risk factors.

1. An in vitro method for predicting a risk of onset of abdominal obesity in a subject, which method comprises the steps of:
a1) measuring the concentration of bacterial 16S rDNA in a biological sample of said subject; and
b) based on the result of the measurement in step a1), determining a risk of onset of abdominal obesity in the subject.

2. The in vitro method according to claim 1, further comprising a step a2) of comparing the bacterial 16S rDNA concentration measured in step a1) with a predetermined value.

3. The in vitro method according to claim 2, wherein a measured concentration of bacterial 16S rDNA in the biological sample of the subject which is higher than the predetermined value is indicative of an increased risk of onset of abdominal obesity within 9 years from the sampling.

4. The in vitro method according to claim 1, wherein the biological sample is selected from the group consisting in blood, serum and plasma sample.

5. The in vitro method according to claim 1, wherein the concentration of bacterial 16S rDNA is measured by real-time PCR.

6. The in vitro method according to claim 1, wherein the subject does not suffer from abdominal obesity at the time of sampling.

7. The in vitro method according to claim 1, wherein the subject does not suffer from general obesity at the time of sampling.

8. The in vitro method according to claim 1, wherein the subject is free of known obesity risk factors and/or known abdominal obesity risk factors selected from the group consisting of age, short sleep duration, early puberty, age at menarche, low activity level, sedentarity, smoking, alcohol intake, hypertension, hypertriglyceridemia, hyperglycemia, genetic and epigenetic factors, familial history of obesity, poor quality of life and poor dietary quality.

9. The in vitro method according to claim 1, wherein the subject is free of a fasting glycemia equal or superior to 6.1 mmol/L or free of the metabolic syndrome.

10. The in vitro method according to claim 1, wherein the subject is 30-65 years old.

11. The in vitro method according to claim 1, wherein the subject displays a plasma baseline C reactive protein concentration lower than 10 mg/l and/or does not present an abundant leukoeyturia and/or does not take antiviral therapy.

12. A method of treatment of a subject predicted to have an increased risk of onset of abdominal obesity comprising the steps of:
a1) measuring the concentration of bacterial 16S rDNA in a biological sample of said subject;
a2) comparing the bacterial 16S rDNA concentration measured in step a1) with a predetermined value;
b) based on the result of the measurement in step a1), determining a risk of onset of abdominal obesity in the subject, wherein a measured concentration of bacterial 16S rDNA in the biological sample of the subject which is higher than the predetermined value is indicative of an increased risk of onset of abdominal obesity within 9 years from the sampling; and
c) if the subject is predicted to have an increased risk of onset of abdominal obesity within 9 years from the sampling, initiating in said subject weight loss treatment.
